(19) United States
(12) Patent Application Publication
(10) Pub. No.: US 2012/0172650 A1
(43) Pub. Date: Jul. 5, 2012

(54) USE OF SOMATOSTATIN OR AN ANALOGUE THEREOF IN COMBINATION WITH EXTERNAL RADIATION THERAPY

(76) Inventors: Laurence Katznelson, Stanford, CA (US); Jane Knox, Stanford, CA (US)

(21) Appl. No.: 13/256,532

(22) PCT Filed: Mar. 17, 2010
(86) PCT No.: PCT/US10/00794

§ 371 Date: Mar. 26, 2012

Related U.S. Application Data
(60) Provisional application No. 61/210,390, filed on Mar. 18, 2009.

(51) Int. Cl. A61N 5/00

(52) U.S. Cl. 600/1

(57) ABSTRACT

Use of somatostatin or analogues thereof to enhance the effects of radiation on cellular proliferation and apoptosis, particularly use of somatostatin combined with externally applied radiation to treat neuroendocrine tumors and/or acromegaly.

### FIELD OF THE INVENTION

The present invention is directed to the use of somatostatin or a somatostatin agonist analog to enhance the effects of external radiation upon cancer cells, particularly for use in patients with neuroendocrine tumors leading to acromegaly.

### BACKGROUND OF THE INVENTION

Acromegaly is an endocrine disorder that is characterized by the excess secretion of growth hormone (GH) and results in excessive growth of bone and soft tissues, multi-system co-morbidities and a heightened risk of premature mortality (Ben-Shlomo, A. et al., Endocrinol. Metab. Clin. North Am., 2001, 30:565-83; Ezzat, S. et al., Medicine (Baltimore), 1994, 73:233-40; Katznelson, L., Growth Horm. IGF Res., 2005, 15 Suppl A:S31-35). Over 90% of all cases of acromegaly are caused by adenomatous growth of pituitary somatotrophic cells. While the preferred treatment for acromegaly is surgical excision, adjuvant medical therapy, including the administration of somatostatin analogs or radiation therapy, is often a necessary course of treatment for this disease. Radiosurgery has been performed on patients with pituitary adenomas for over 50 years.

Studies in which patients received both a somatostatin analog (octreotide) and external radiation suggest that the two treatments are best administered separately. Landolt et al. (J. Clin. Endocrinol. Metab., 2000, 85:1287-9) and Pollock et al. (J. Neurosurg., 2002, 97:525-30) both propose that somatostatin analogs may be radio-protective in acromegaly. Landolt et al. observed that fewer octreotide-treated acromegaly patients receiving stereotactic radiosurgery reached normal GH and IGF levels than did acromegaly patients receiving radiation therapy alone. From these studies, Landolt et al. concluded that octreotide actually protected pituitary adenomas of the acromegalic patients from the beneficial effects of stereotactic radiosurgery. These findings led Landolt et al. to recommend administering stereotactic radiosurgery only during a gap in octreotide treatments. Pollock et al. concluded that the absence of hormone suppressive medicines, such as somatostatin or a somatostatin analog coupled with maximum radiation doses, correlated with an endocrine cure. In the Pollack study, the authors report that no disease cure was obtained in those patients receiving pituitary hormone-suppressive medications at the time of radiosurgery.

These findings raise the issue of whether or not treatment with somatostatin analogs should be stopped prior to or during courses of radiation therapy.

### SUMMARY OF THE INVENTION

This invention is directed to the combined use of somatostatin, a somatostatin analog, or pharmaceutically acceptable salts thereof and externally administered radiation as a means for enhancing the effects of said radiation in treating cancer. Successful treatment of a cancer by the combination of a somatostatin, a somatostatin analog, or pharmaceutically acceptable salts thereof and externally administered radiation may be evidenced by tumor shrinkage, delayed tumor growth, decreased cancer cellular proliferation, decreased cancer cellular survival, cell cycle arrest and/or increased cancer cell death (apoptosis) as well as alleviation of excess hormone production and/or alleviation of any other biological complications resulting from the tumor, it's growth and it's effects upon surrounding and distant tissues.

In one aspect, the present invention is directed to the use of somatostatin, a somatostatin analog, or pharmaceutically acceptable salts thereof, to enhance the effects of externally administered radiation upon cancer cells. In one embodiment the cancer cell resides in vivo in a subject in need thereof. In one embodiment, the subject is a mammal such as a human. In one embodiment, the subject suffers from a neuroendocrine tumor. In another embodiment, the neuroendocrine tumor is a pituitary adenoma. The subject suffering from the pituitary adenoma may be an acromegalic.

In another aspect, the present invention is directed to the use of somatostatin, a somatostatin analog, or pharmaceutically acceptable salts thereof, to enhance the effects of externally administered radiation upon tumor shrinkage, delayed tumor growth, decreased cancer cellular proliferation, decreased cancer cellular survival, increased cell cycle arrest and/or increased cancer cell death (apoptosis or necrosis). A preferred enhanced effect is an increase in the apoptotic death of cancer cells.

In one embodiment, the somatostatin, somatostatin analog, or pharmaceutically acceptable salts thereof, is administered prior to externally administered radiation therapy. The administration may be made in advance of externally administered radiation, such as years or months in advance, or just prior to radiation therapy, such as weeks or days, such as from about 1 day to 7 days, or hours in advance, such as from about 48 hours, 24 hours or even 0 hours in advance (i.e., immediately before radiation). In one embodiment, the exposure to the somatostatin, somatostatin analog, or pharmaceutically acceptable salts thereof, is 48 hours in advance of external radiation application. In one embodiment, the somatostatin, somatostatin analog or pharmaceutically acceptable salts thereof, is administered in conjunction with externally administered radiation therapy. The administration may also be made after externally administered radiation, such as years or months after, or just after radiation therapy, such as weeks or days, such as from about 1 day to 7 days after, or hours after, such as from about 48 hours, 24 hours or even 0 hours after (i.e., immediately after radiation).

The somatostatin analog useful in the practice of the instant invention is any analog which acts as a somatostatin agonist. The somatostatin analog may be a peptide analog or a small molecule analog. In one embodiment, the analog is a somatostatin type-1, somatostatin type-2, somatostatin type-3, somatostatin type-4 or somatostatin type-5 agonist. In another embodiment, the analog is a somatostatin type-1, somatostatin type-2, somatostatin type-3, somatostatin type-4 or somatostatin type-5 selective agonist. In yet another embodiment, the analog may bind to a combination of any 2 or more somatostatin type-1, somatostatin type-2, somatostatin type-3, somatostatin type-4 or somatostatin type-5 receptors. In yet another embodiment, the analog may bind selectively to a combination of any 2 or more somatostatin type-1, somatostatin type-2, somatostatin type-3, somatostatin type-4 or somatostatin type-5 receptors. In yet a further embodiment, the selectively binding somatostatin agonist analog is a pansomatostatin.

In one embodiment, the analog is a somatostatin type-2 receptor agonist or a selective somatostatin type-2 receptor agonist. Exemplary somatostatin type-2 selective receptor agonist analogs include lanreotide, octreotide, vapreotide and the like.

There are several advantages to the combination of externally administered radiation and somatostatin agonist treatment. The current standard of care requires the cessation of somatostatin treatment for a minimum of 6 months prior to administering external radiation. The combination of external radiation and somatostatin agonist treatments described herein would alleviate this need to stop somatostatin agonist treatments and would transform the current treatment paradigm. As demonstrated herein, cancer cells receiving both a somatostatin analog and external radiation exhibit increased apoptosis. This increase in the death of irradiated cancer cells may allow for the administration of lower doses of radiation to patients, with the added benefit of reduced damage to the cells and tissues surrounding the target tumor. It is envisioned that the combination therapy proposed herein will offer patients a less toxic and more efficacious means of treatment for any cancer which is somatostatin-sensitive and can be subjected to externally applied radiation. Neuroendocrine cancers such as pituitary adenomas are one type of cancer which may benefit from the combined treatment of externally administered radiation and a somatostatin analog. Pituitary adenomas which may benefit from the combined treatment of externally administered radiation and a somatostatin analog include ACTH-secreting adenomas, prolactin secreting adenomas, GH secreting adenomas and non-GH-secreting adenomas.

Other features and advantages of the invention become more apparent on reading the following description of embodiments of the invention given by way of non-limiting example.

### BRIEF DESCRIPTION OF THE DRAWINGS

FIG. 1. The dose response of GH3 cells to lanreotide (A) and gamma irradiation (B). GH3 cells were plated in Petri dishes in triplicate and treated with various doses of lanreotide or radiation. Cells were incubated for 21 days for colony formation. Data are presented as surviving fraction against dose on a log-linear plot. Data points represent the mean±S.D. of three samples per dose point.

FIG. 2. Radiation survival curves for GH3 cells treated with irradiation alone or in the presence of lanreotide at doses of 100 nm or 1000 nM. Lanreotide was given 48 hours or 24 hours prior to or immediately (0 hour) before radiation. The total exposure time for lanreotide was 48 hours. (B) Radiation survival of GH3 cells treated with 10 Gy radiation with or without 100 nM lanreotide. Data represent the mean±S.D. from two experiments. *p<0.01 compared with 10 Gy radiation alone.

FIG. 3. Comparison of cell cycle redistribution of GH3 cells after irradiation in the absence or presence of lanreotide. Lanreotide at 100 nM was added to the media either 48 hours, 24 hours or immediately (0 hour) before radiation and kept in the media until collection of samples. The cell cycle was analyzed using FACScan flow cytometer. (A) Cell cycle histogram from FASCan flow cytometer. (B) Percent of cells in sub-G1 phase of the cell cycle after irradiation (0-168 hours). (C) Accumulated apoptosis that was estimated by calculating the area under the curves in FIG. 3B. *p<0.05 compared with radiation alone.

FIG. 4. Dose response of GH3 xenograft tumors to lanreotide. GH3 tumor-bearing mice were injected with 2.5, 5, 10, 20 or 50 mg/kg lanreotide daily for 5 days. The 4× tumor growth delay (TGD) times were calculated for each tumor and averaged for each dose point. Data are presented as TGD in days as a function of dose. Data points represent the mean±standard deviation of two experiments.

FIG. 5. Tumor growth curves of mice with GH3 tumors treated with lanreotide at doses of 2.5, 5, or 10 mg/kg once daily (qd) or twice daily (bid). Data are presented as the average tumor volume of each group (mean±standard deviation) versus time from start of treatment.

FIG. 6. Tumor growth curves of GH3 tumors in mice after combined treatment with lanreotide and fractionated local tumor radiation. Lanreotide was injected subcutaneously at 10 mg/kg daily for 5 days. Radiation was delivered locally to the tumors at a daily dose of 250, 200, or 150 cGy for 5 days. There were four groups in each study: 1) untreated control (open square); 2) lanreotide alone (solid circle); 3) radiation alone (solid diamond); and 4) combination of lanreotide and radiation (solid triangle). Six to eight animals were used in each group. Data are presented as the average tumor volume of each group (mean±standard deviation) versus time from start of treatment.

FIG. 7. Tumor growth curves of mice with GH3 tumors treated with lanreotide and fractionated radiation. Lanreotide was injected at a dose of 10 mg/kg daily for 10 days. Radiation was delivered locally to the tumors at a daily dose of 150 cGy for 5 days. There were four groups: 1) untreated control (open square); 2) lanreotide alone (solid circle); 3) radiation alone (solid diamond); and 4) combination of lanreotide and radiation (solid triangle). Six animals were used in each group. Data are presented as the average tumor volume of each group (mean±standard deviation) versus time from start of treatment.

### DETAILED DESCRIPTION OF THE INVENTION

Somatostatin, a tetradecapeptide discovered by Brazeau et al. (Science, 1973, 179:77-79), has been shown to have potent inhibitory effects on various secretory processes and cell proliferation in normal and neoplastic human tissues such as pituitary, pancreas and the gastrointestinal tract. Somatostatin also acts as a neuromodulator in the central nervous system. These biological effects of somatostatin, all inhibitory in nature, are elicited through a series of G protein coupled receptors, of which five different subtypes have been characterized, hereinafter referred to as “SSTR1”, “SSTR2”, “SSTR3”, “SSTR4” and “SSTR5” for each of the five receptors or generally and/or collectively as “SSTR” (Patel, Y. C., Front. Neuroendocrinol., 1999, 20:157-98; and Zatelli, M. C. et al., J. Endocrinol. Invest., 2004, 27 Suppl(6):168-70). These five subtypes have similar affinities for the endogenous somatostatin ligands but have differing distribution in various tissues. Somatostatin binds to the five distinct receptor subtypes with relatively high and equal affinity for each.

There is evidence that somatostatin regulates cell proliferation by arresting cell growth via SSTR1, 2, 4 and 5 receptor subtypes (Buscail, L. et al., Proc. Natl. Acad. Sci. USA, 1995, 92:1580-4; Buscail, L. et al., Proc. Natl. Acad. Sci. USA, 1994, 91:2315-9; Florio, T. et al., Mol. Endocrinol., 1999, 13:24-37; and Sharma, K. et al., Mol. Endocrinol., 1999, 13:82-90) or by inducing apoptosis via the SSTR3 receptor subtype (Sharma, K. et al., Mol. Endocrinol., 1996, 10:1688-96). Somatostatin and various analogues have been shown to inhibit normal and neoplastic cell proliferation in vitro and in vivo (Lamberts, S. W. et al., Endocrin. Rev., 1991, 12:450-82) via specific somatostatin receptors (Patel, Y. C., Front Neuroendocrin., 1999, 20:157-98), possibly via different postreceptor actions (Weckbecker, G. et al., Pharmacol. Ther., 1993, 60:245-64; Bell, G. I. and Reisine, T., Trends Neurosci., 1993, 16:34-8; Patel, Y. C. et al., Biochem. Biophys. Res. Comm., 1994, 198:605-12; and Law, S. F. et al., Cell Signal, 1995, 7:1-8). In addition, there is evidence that distinct SSTR subtypes are expressed in normal and neoplastic human tissues (Virgolini, I. et al., Eur. J. Clin. Invest., 1997, 27:645-7) conferring different tissue affinities for various somatostatin analogues and variable clinical response to their therapeutic effects.

Binding to the different types of SSTR subtypes has been associated with the treatment of various conditions and/or diseases. For example, the inhibition of growth hormone has been attributed to SSTR2 (Raynor, et al., Molecular Pharmacol., 1993, 43:838; and Lloyd, et al., Am. J. Physiol., 1995, 268:G102) while the inhibition of insulin has been attributed to SSTR5. Activation of SSTR2 and SSTR5 has been associated with growth hormone suppression and more particularly GH secreting adenomas (acromegaly) and TSH secreting adenomas. Activation of SSTR2 but not SSTR5 has been associated with treating prolactin secreting adenomas.

It has long been known that many types of cancers are characterized by abnormal levels of somatostatin receptor molecules (Reubi, J. C., et al., Eur. J. Nucl. Med., 2001, 28:836-846). There is general agreement that most tumors typically express at least one type of somatostatin receptor and that varying densities of SSTRs may be expressed in the cells contained within a particular tumor. Reubi and Landolt (J. Clin. Endo. Metab., 1984, 59:1149-1151) surveyed human pituitary adenomas from five acromegalic patients and demonstrated the presence of a large number of saturable and high affinity binding sites for the somatostatin analog SMS 201-995 (octreotide; SEQ ID NO:2).

The availability of cloned SSTR subtype genes has allowed somatostatin analogs to be characterized by their affinities for the five receptor types and these studies have revealed considerable variability in SSTR subtype specificity among somatostatin analogs (Raynor, et al., Molecular Pharmacol., 1993, 43:838-844; Patel, et al. TEM, 1997, 8:398-404). SSTR2 type somatostatin analogs were and are most readily available for such studies however other studies using SSTR1, SSTR3, SSTR4 and/or SSTR5 analogs have been carried out as well. Examples of cancers which have been identified to express abnormal levels, i.e., an overabundance of SSTR receptors of any type as compared to normal tissues, include but are not limited to: GH secreting pituitary adenomas, inactive pituitary adenomas and endocrine gastroenteropancreatic (GEP) tumors (see Schaer, J-C., et al., Int. J. Cancer, 1997, 70:530-537) and paragangliomas, pheochrymocytomas, medullary thyroid carcinomas (MTC) and malignant lymphomas (see Reubi, J. C., et al., Metabolism, 1992, 41:104-110). These SSTR-bearing tumors express SSTR2 and SSTR5 most frequently, with SSTR3 and SSTR4 occurring less frequently.

Other cancers and tumors expressing or overexpressing somatostatin receptors include meningiomas, neuroblastomas and mesenchymal tumors. Prostate cancers (see Reubi, J. C., et al., Yale J. Biol. Med., 1997, 70:471-479; Vainas, J. G., Chemotherapy, 2001, 47:109-126); Koutsilieris, M., et al., Clin. Cancer Res., 2004, 10:4398-4405), small cell lung cancer (Prevost, G. et al., Life Sci., 1994, 55:155-162; Bombardieri, E., et al., Eu. J. Cancer, 1995, 31A:184-188) and malignancies of the breast (Weckbecker, G., et al., Cancer Res., 1992, 52:4973-4978; Ingle, J. N., et al., Invest. New Drugs, 1996, 14:235-237) are examples of solid tumors which have been shown to be responsive to somatostatin analogs as evidenced by a decrease in growth. Nasopharyngeal cancer (Loh, K. S., et al., Virchows Arch., 2002, 441:444-448) and medulloblastomas (Cervera, P. et al., J. Neuroendocrinol., 2002, 14:458-471) have also been found to overexpress somatostatin receptors. One group reports that SSTR3 is expressed at very high levels in almost all human tumors (Virgolini, Eur. J. Clin. Invest., 1997, 27:793-800).

In addition to the finding that various somatostatin receptors are located on cells in tumor tissue, it has also been shown that somatostatin receptors are located on cells of tissues that support tumor tissue and aid tumor growth. Denzler, B. et al., (Cancer, 1999, 85:188-198) demonstrate that the SSTR2 somatostatin agonist octreotide localizes to peritumoral veins and tumor beds that surround and support tumor tissue. Somatostatin receptors have been localized to tissues supporting gastric carcinomas, breast carcinomas, renal cell carcinomas, prostate carcinomas, endometrial carcinomas, pancreatic adenocarcinomas, parathyroid adenomas, MTC, soft tissue tumors, melanomas and surrounding lymph nodes, bone and lung metastases.

Lanreotide (D-2-Nal-Cys-Tyr-D-Trp-Lys-Val-Cys-Thr-NH₂; sold as Somatuline® by IPSEN Pharma; SEQ ID NO:1) and octreotide (H-D-Phe-Cys-Phe-D-Trp-Lys-Thr-Cys-Thr-ol; sold as Sandostatin® by Novartis AG Corporation; SEQ ID NO:2) are two well known somatostatin analogs which are approved in the U.S. and Europe for the treatment of acromegaly and for the control of symptoms associated with VIPomas and metastatic carcinoid tumors. Each analog binds preferentially to the SSTR2 receptor and, to a lesser degree, to the SSTR5 type receptor. A third well-known, but lesser used somatostatin analog, is vapreotide having the sequence D-Phe-Cys-Tyr-D-Trp-Lys-Val-Cys-Trp-NH₂ (sold as Sanvar® by Debiovision, Inc.; SEQ ID NO:3). It is preferred to have an analog which is selective for the specific somatostatin receptor subtype or subtypes responsible for the desired biological response, thus reducing interaction with other receptor subtypes which could lead to undesirable side effects.

A large body of literature exists relating to clinical uses of octreotide and lanreotide. As summarized in Lamberts et al. (New England J. of Med., 1996, 334:246-254) octreotide has been investigated for use in treating thyrotropin-secreting pituitary adenomas, nonsecretory pituitary adenomas, and corticotropin-secreting pituitary adenomas such as bronchial and thymic carcinoids, medullary thyroid carcinomas and pancreatic islet cell tumors, but not those not associated with Cushing's disease. According to Lamberts et al. octreotide treatment only occasionally resulted in transient inhibition of tumor growth. Lamberts et al. further disclose that octreotide has been studied for use in gastrointestinal and pancreatic diseases but with variable results: octreotide was not effective in treating bleeding from peptic ulcers but was effective in controlling bleeding from esophageal varices. Lamberts et al. describes octreotide as being ineffective in the treatment of acute pancreatitis but efficacious in reducing fluid production by pancreatic fistulas and pseudocysts. Clinical trials of octreotide for treatment of watery diarrhea in AIDS patients were also described in Lamberts et al. Woltering et al. (Investigational New Drugs, 1997, 15:77-86) discuss the investigation of octreotide as an anti-angiogenic agent.

Lanreotide has been applied to surgical wounds induced in tumor-implanted mice to study its effect on wound-induced acceleration of tumor growth and it has been suggested as a useful endocrine anti-secretogogue in cyto-reductive cancer treatment (see Bogden et al., Brit. J. Cancer, 1997, 75:1021-1027). Wasko et al., (Neuro. Endocrinol. Lett., 2003, 24:334-338) show that treatment with lanreotide induced apoptosis of endocrine tumor cells. Colao et al., (J. Clin. Endo. Metab., 2006, 91:2112-2118) describe the successful shrinkage of tumors in lanreotide treated patients newly diagnosed with acromegaly. Using a rat hepatocellular carcinoma model system, it has been shown that lanreotide significantly decreased the size of induced preneoplastic foci in vitro (Borbath, I. et al., Cancer Sci., 2007, 98:1931-1839). In patients suffering from type 1 gastric carcinoid tumors, lanreotide has also been shown to effectively reduce tumor load, both in number and in size, with a concomitant decrease in serum gastrin levels (Grozinsky-Glasberg, S. et al., Eur. J. Endo., 2008, 159:475-482)

In addition to the commercially available octreotide and lanreotide, a large number of second generation somatostatin analogs have been proposed for use as therapeutic agents to detect and/or treat cancer and other somatostatin-responsive disease states. Such second generation somatostatin analogs are described in:

| U.S. Pat. No. 3,997,517 | U.S. Pat. No. 4,000,259 | U.S. Pat. No. 4,011,182 |
|---|---|---|
| U.S. Pat. No. 4,077,952 | U.S. Pat. No. 4,098,782 | U.S. Pat. No. 4,100,153 |
| U.S. Pat. No. 4,104,267 | U.S. Pat. No. 4,105,603 | U.S. Pat. No. 4,115,554 |
| U.S. Pat. No. 4,122,077 | U.S. Pat. No. 4,123,425 | U.S. Pat. No. 4,130,554 |
| U.S. Pat. No. 4,133,782 | U.S. Pat. No. 4,139,526 | U.S. Pat. No. 4,140,767 |
| U.S. Pat. No. 4,145,337 | U.S. Pat. No. 4,146,612 | U.S. Pat. No. 4,159,263 |
| U.S. Pat. No. 4,161,521 | U.S. Pat. No. 4,162,248 | U.S. Pat. No. 4,185,010 |
| U.S. Pat. No. 4,190,575 | U.S. Pat. No. 4,190,648 | U.S. Pat. No. 4,191,754 |
| U.S. Pat. No. 4,204,990 | U.S. Pat. No. 4,209,426 | U.S. Pat. No. 4,211,693 |
| U.S. Pat. No. 4,215,039 | U.S. Pat. No. 4,224,190 | U.S. Pat. No. 4,235,886 |
| U.S. Pat. No. 4,238,481 | U.S. Pat. No. RE30,548 | U.S. Pat. No. 4,261,885 |
| U.S. Pat. No. 4,280,953 | U.S. Pat. No. 4,282,143 | U.S. Pat. No. 4,291,022 |
| U.S. Pat. No. 4,310,518 | U.S. Pat. No. 4,316,890 | U.S. Pat. No. 4,316,891 |
| U.S. Pat. No. 4,328,214 | U.S. Pat. No. 4,358,439 | U.S. Pat. No. 4,360,516 |
| U.S. Pat. No. 4,369,179 | U.S. Pat. No. 4,395,403 | U.S. Pat. No. 4,427,661 |
| U.S. Pat. No. 4,428,942 | U.S. Pat. No. 4,435,385 | U.S. Pat. No. 4,853,731 |
| U.S. Pat. No. 4,485,101 | U.S. Pat. No. 4,486,415 | U.S. Pat. No. 4,505,897 |
| U.S. Pat. No. 4,508,711 | U.S. Pat. No. 4,522,813 | U.S. Pat. No. 4,585,755 |
| U.S. Pat. No. 4,603,120 | U.S. Pat. No. 4,611,054 | U.S. Pat. No. 4,612,366 |
| U.S. Pat. No. 4,650,787 | U.S. Pat. No. 4,663,435 | U.S. Pat. No. 4,684,620 |
| U.S. Pat. No. 4,703,034 | U.S. Pat. No. 4,725,577 | U.S. Pat. No. 4,728,638 |
| U.S. Pat. No. 4,803,261 | U.S. Pat. No. 4,816,438 | U.S. Pat. No. 4,837,303 |
| U.S. Pat. No. 4,853,371 | U.S. Pat. No. 4,871,717 | U.S. Pat. No. 4,897,445 |
| U.S. Pat. No. 4,904,642 | U.S. Pat. No. 5,059,653 | U.S. Pat. No. 5,506,339 |
| U.S. Pat. No. 5,552,520 | U.S. Pat. No. 5,595,760 | U.S. Pat. No. 5,597,894 |
| U.S. Pat. No. 5,620,675 | U.S. Pat. No. 5,633,263 | U.S. Pat. No. 5,650,006 |
| U.S. Pat. No. 5,693,679 | U.S. Pat. No. 5,700,905 | U.S. Pat. No. 5,708,135 |
| U.S. Pat. No. 5,716,596 | U.S. Pat. No. 5,770,687 | U.S. Pat. No. 5,776,894 |
| U.S. Pat. No. 5,783,170 | U.S. Pat. No. 5,804,157 | U.S. Pat. No. 5,811,392 |
| U.S. Pat. No. 5,814,298 | U.S. Pat. No. 5,820,845 | U.S. Pat. No. 5,830,431 |
| U.S. Pat. No. 5,833,942 | U.S. Pat. No. 5,843,401 | U.S. Pat. No. 5,843,903 |
| U.S. Pat. No. 5,846,934 | U.S. Pat. No. 5,871,711 | U.S. Pat. No. 5,874,529 |
| U.S. Pat. No. 5,883,293 | U.S. Pat. No. 5,925,618 | U.S. Pat. No. 5,929,209 |
| U.S. Pat. No. 5,932,189 | U.S. Pat. No. 5,955,426 | U.S. Pat. No. 5,965,108 |
| U.S. Pat. No. 5,965,694 | U.S. Pat. No. 5,972,308 | U.S. Pat. No. 5,981,477 |
| U.S. Pat. No. 5,985,241 | U.S. Pat. No. 6,001,801 | U.S. Pat. No. 6,001,960 |
| U.S. Pat. No. 6,017,509 | U.S. Pat. No. 6,017,512 | U.S. Pat. No. 6,020,349 |
| U.S. Pat. No. 6,025,372 | U.S. Pat. No. 6,051,206 | U.S. Pat. No. 6,051,554 |
| U.S. Pat. No. 6,057,338 | U.S. Pat. No. 6,063,796 | U.S. Pat. No. 6,083,960 |
| U.S. Pat. No. 6,117,880 | U.S. Pat. No. 6,127,343 | U.S. Pat. No. 6,183,722 |
| U.S. Pat. No. 6,214,316 | U.S. Pat. No. 6,225,284 | U.S. Pat. No. 6,241,965 |
| U.S. Pat. No. 6,262,229 | U.S. Pat. No. 6,265,375 | U.S. Pat. No. 6,316,004 |
| U.S. Pat. No. 6,316,414 | U.S. Pat. No. 6,329,389 | U.S. Pat. No. 6,352,982 |
| U.S. Pat. No. 6,355,613 | U.S. Pat. No. 6,358,491 | U.S. Pat. No. 6,387,932 |
| U.S. Pat. No. 6,407,059 | U.S. Pat. No. 6,440,933 | U.S. Pat. No. 6,465,613 |
| U.S. Pat. No. 6,468,974 | U.S. Pat. No. 6,586,445 | U.S. Pat. No. 6,602,849 |
| U.S. Pat. No. 6,635,647 | U.S. Pat. No. 6,703,481 | U.S. Pat. No. 6,727,269 |
| U.S. Pat. No. 6,759,415 | U.S. Pat. No. 6,777,408 | U.S. Pat. No. 6,818,739 |
| U.S. Pat. No. 6,852,725 | U.S. Pat. No. 6,864,234 | U.S. Pat. No. 6,872,827 |
| U.S. Pat. No. 6,903,074 | U.S. Pat. No. 6,930,088 | U.S. Pat. No. 6,943,145 |
| U.S. Pat. No. 7,019,004 | U.S. Pat. No. 7,074,775 | U.S. Pat. No. 7,084,244 |
| U.S. Pat. No. 7,094,753 | U.S. Pat. No. 7,109,166 | U.S. Pat. No. 7,115,566 |
| U.S. Pat. No. 7,115,634 | U.S. Pat. No. 7,122,622 | U.S. Pat. No. 7,144,859 |
| U.S. Pat. No. 7,189,856 | U.S. Pat. No. 7,202,330 | U.S. Pat. No. 7,220,765 |
| U.S. Pat. No. 7,238,695 | U.S. Pat. No. 7,312,304 | U.S. Patent Publication |
| | | No. 20020052315; |
| U.S. Patent Publication | U.S. Patent Publication | U.S. Patent Publication |
| No. 20020089576; | No. 20020151501; | No. 20030114362; |
| U.S. Patent Publication | U.S. Patent Publication | U.S. Patent Publication |
| No. 20030120072; | No. 20030144186; | No. 20030153494; |
| U.S. Patent Publication | U.S. Patent Publication | U.S. Patent Publication |
| No. 20030191134; | No. 20030211981; | No. 20040006089; |
| U.S. Patent Publication | U.S. Patent Publication | U.S. Patent Publication |
| No. 20040019069; | No. 20040019092; | No. 20040023315; |
| U.S. Patent Publication | U.S. Patent Publication | U.S. Patent Publication |
| No. 20040038970; | No. 20040044177; | No. 20040082501; |
| U.S. Patent Publication | U.S. Patent Publication | U.S. Patent Publication |
| No. 20040097418; | No. 20040102364; | No. 20040110779; |
| U.S. Patent Publication | U.S. Patent Publication | U.S. Patent Publication |
| No. 20040171530; | No. 20040176379; | No. 20040181032; |
| U.S. Patent Publication | U.S. Patent Publication | U.S. Patent Publication |
| No. 20040209798; | No. 20040209908; | No. 20040242842; |
| U.S. Patent Publication | U.S. Patent Publication | U.S. Patent Publication |
| No. 20050008573; | No. 20050070470; | No. 20050090429; |
| U.S. Patent Publication | U.S. Patent Publication | U.S. Patent Publication |
| No. 20050154039; | No. 20050164922; | No. 20050226813; |
| U.S. Patent Publication | U.S. Patent Publication | U.S. Patent Publication |
| No. 20050239796; | No. 20050245571; | No. 20060063704; |
| U.S. Patent Publication | U.S. Patent Publication | U.S. Patent Publication |
| No. 20060069017; | No. 20060211607; | No. 20060223826; |
| U.S. Patent Publication | U.S. Patent Publication | U.S. Patent Publication |
| No. 20060258572; | No. 20060258838; | No. 20070032653; |
| U.S. Patent Publication | U.S. Patent Publication | U.S. Patent Publication |
| No. 20070041902; | No. 20070043095; | No. 20070129313; |
| U.S. Patent Publication | U.S. Patent Publication | U.S. Patent Publication |
| No. 20070129422; | No. 20070161045; | No. 20070259811; |
| EP Patent 0 143 307 | EP Patent 0 389 180 | EP Patent 0 714 911 |
| EP Application 0 030 920 | EP Application 0 505 680 | EP Application 0 083 305 |
| A2 | A2 | A2 |
| EP Application 0 203 031 | EP Application 0 222 578 | EP Application 0 363 589 |
| A2 | A2 | A2 |
| WO 97.01579 | WO 91/18016 | WO 91/09056 |
| WO 91/12811 | WO 88/05052 | U.K. Application GB |
| | | 2,095,261 |
| French Application FR | | |
| 2,522,655 | | |

Radiation is a therapeutic treatment used to treat many types of cancer; along with chemotherapy and surgery, radiation is used in approximately 60% of treatment regimens. For particular cancers such as basal cell carcinomas of the skin, head and neck, prostate cancers and bladder cancers, radiation, in any of several forms, is used as the primary therapy. Radiation therapy encompasses both local and total body administration and is delivered in various ways depending on the type(s) of cancer, the location(s) of the diseased tissue and the level(s) to which the cancer has developed and/or spread in the subject.

The cytotoxic effect of radiation upon a cell arises from the ability of the radiation to cause one or more breaks in one or both strands of the various DNA molecules inside the cell. Cells in all phases of the cell cycle are susceptible to this effect. Healthy cells with functioning cell cycle check proteins and repair enzymes are far more likely to be able to repair radiation damage and return to normal functions. The DNA damage sustained by neoplastic cancerous cells is more lethal because the cellular mechanisms are less capable of repairing the damage.

Tumors and tissues themselves are also characterized by a range of susceptibilities to radiation therapy; lymphoma and leukemia are very sensitive to radiation therapy, while renal cancer and gland tumors are fairly insensitive. A tumor that is considered radiosensitive is one which can be eradicated by a dose(s) of radiation that is also well tolerated by the surrounding tissues. Unsurprisingly, different tissue types within the body tolerate radiation at different doses. Tissues that undergo frequent cell division are most affected by radiation; these same tissues are often similarly sensitive to cell cycle specific chemotherapy agents. Sources of radiation include: Americium, chromic phosphate, radioactive, Cobalt, ¹³¹I-ethiodized oil, gold (radioactive, colloidal) iobenguane, radium, radon, sodium iodide (radioactive), sodium phosphate (radioactive) and others.

The presence or lack of oxygen in a tumor tissue also affects the sensitivity of that tissue to radiation. The interior mass of a tumor, particularly a large tumor, may lack oxygen rendering the tumor hypoxic. Hypoxic tumors can be 2-3 times less responsive to radiation treatment than non-hypoxic tumors. Certain agents used in conjunction with radiation treatment, such as some of the radiosensitizing agents, work by increasing the singlet oxygen species in the vicinity of the tumor and therefore increasing its radiosensitivity. Other compounds used in conjunction with radiation therapy include radioprotectants which are designed to protect surrounding tissue from some of the effects of radiation therapy.

Typically, radiation therapy is administered in pulses over a period of time of about 1 to about 2 weeks however treatment may be administered for longer periods of time. Examples of radiation therapies include conformal radiation therapy, coronary artery brachytherapy, fast neutron radiotherapy, intensity modulated radiotherapy (IMRT), interoperative radiotherapy, interstitial brachytherapy, interstitial breast brachytherapy, organ preservation therapy and steriotactic radiosurgery.

Radiation therapy itself can be classified according to two primary types, internal and external radiation therapy. External therapy involves the administration of radiation via a machine capable of producing high-energy external beam radiation. This therapy can include either total body irradiation or can be localized to the region of the tumor. The radiation itself can be either electromagnetic (X-ray or gamma radiation) or particulate (α or β particles). Radiation administered by external means include external beam radiation such as cobalt therapy and can include other forms of ionizing radiation such as X-rays, γ-rays, β-rays, ultraviolet light, near ultra-violet light and other sources of radiation including, for example, π-mesons. The treatment requirements will differ depending upon the characteristics of the tumor. External radiation is often used pre- or post-operatively; either to shrink the tumor before surgery or to eliminate any cancer cells remaining after surgery.

Internal radiation therapy, also termed brachytherapy, involves implantation of a radioactive isotope as the source of the radiation. There are a variety of methods of delivering internal radiation sources, including but not limited to, permanent, temporary, sealed, unsealed, intracavity or interstitial implants. The choice of implant is determined by a variety of factors including the location and extent of the tumor. Internally delivered radiation includes therapeutically effective radioisotopes injected into a patient. Such radioisotopes include, but are not limited to, radionuclide metals such as ¹⁸⁶RE, ¹⁸⁸RE, ⁶⁴Cu, ⁹⁰ytrium, ¹⁰⁹Pd, ²¹²Bi, ²⁰³Pb, ²¹²Pb, ²¹¹At, ⁹⁷Ru, ¹⁰⁵Rh, ¹⁹⁸Au, ¹⁹⁹Ag and ¹³¹I. These radioisotopes generally will be bound to carrier molecules when administered to a patient.

A sub-category of internal radiation is radioimmunotherapy. Radioimmunotherapy offers targeted, internal administration of radiation by the use of monoclonal antibodies. Monoclonal antibodies (MABs) are a class of antibodies which target specific cell types by recognizing and binding to specific targets found on cell surfaces. When raised against cancerous cells, MABs target those cells within a host system; the attachment of radioisotopes to MABs thus allows for an internal radiation scheme which targets those cells recognized by the antibodies.

The side effects of radiation are similar to those of chemotherapy and arise for the same reason: damage of healthy tissue. Radiation therapy is generally more localized than chemotherapy, but treatment is still accompanied by damage to previously healthy tissue. Common side effects of radiation include: bladder irritation, fatigue, diarrhea, low blood counts, mouth irritation, taste alteration, loss of appetite, alopecia, skin irritation, change in pulmonary function, enteritis, sleep disorders and others. Radiation also shares with chemotherapy the disadvantage of being mutagenic, carcinogenic and teratogenic. While normal cells usually begin to recover from treatment within two hours of treatment, mutations may be induced in the genes of the healthy cells. These risks are elevated in certain tissues, such as those in the reproductive system. It has also been found that different patients will tolerate radiation differently. Doses that may not lead to new cancers in one individual may in fact spawn additional cancers in another individual. This could be due to pre-existing mutations in cell cycle check proteins or repair enzymes, but current practice is not be able to easily predict to which individual and what dose poses a risk.

To study the effects of somatostatin analogs and external radiation on tumor proliferation, it would be optimal to utilize human GH producing pituitary adenoma cells. Dispersed human pituitary tumor cells, however, are difficult to maintain for prolonged passages in culture (Danila, D. C. et al., J. Clin. Endocrinol. Metab., 2001, 86:2976-81; Danila, D. C. et al., J. Clin. Endocrinol. Metab., 2000, 85:1180-7). Because it is difficult to investigate prolonged anti-proliferative effects in human pituitary tumor cells in vitro, other cell and tissue culture systems are often used. Some studies have successfully utilized human fetal pituitary cells (Shimon, I. et al., J. Clin. Invest., 1997, 789-98) though this model is not representative of an adenoma cell line. Rat pituitary tumor GH3 cells are a particularly useful model system as these cells express somatostatin receptors and are somatostatin responsive (Dasgupta, P. et al., Biochem. Biophys. Res. Comm., 1999, 259:379-84). In a colony formation assay, lanreotide exhibited a dose-related anti-proliferative effect (IC50=57 nM) when applied to GH3 cells.

For patients with acromegaly, particularly for those patients with incomplete surgical resections, somatostatin analogs are often used as adjuvant therapeutic agents (AACE Medical Guidelines for Clinical Practice for the diagnosis and treatment of acromegaly, Endocr. Pract., 2004, 10:213-25). Radiation therapy is also utilized as adjuvant therapy for persistent, active disease (Castinetti, F. et al., J. Clin. Endocrinol. Metab., 2005, 90:4483-8). Because patients are often symptomatic at the time of radiation therapy, somatostatin analogues are often administered in conjunction with radiation.

As discussed previously, Landolt et al. and Pollack et al. raise concerns that the anti-proliferative effects of somatostatin analogues may protect cancer cells from the tumoricidal effects of external irradiation. Thus, the question as to whether somatostatin analogues should be withheld at the time of radiation therapy remains controversial.

### Nomenclature and Abbreviations

Unless defined otherwise, all technical and scientific terms used herein have the same meaning as commonly understood by one of ordinary skill in the art to which this invention belongs. Also, all publications, patent applications, patents and other references mentioned herein are incorporated by reference in their entirety.

A “subject”, as used herein and throughout this application, refers to a mammalian or non-mammalian animal including, for example and without limitation, a human, a rat, a mouse or farm animal. Reference to a subject does not necessarily indicate the presence of a disease or disorder. The term “subject” includes, for example, a mammalian or non-mammalian animal being dosed with somatostatin or a somatostatin agonist analog with or without radiation as part of an experiment, a mammalian or non-mammalian animal being treated to help alleviate a disease or disorder, and a mammalian or non-mammalian animal being treated prophylactically to retard or prevent the onset of a disease or disorder. Subject mammals may be human subjects of any age, such as an infant, a child, an adult or an elderly adult.

A “therapeutically acceptable amount” of a compound, composition or dosage of radiation and somatostatin or somatostatin analog of the invention, regardless of the formulation or route of administration, is that amount which elicits a desired biological response in a subject. The biological effect of the therapeutic amount may occur at and be measured at many levels in an organism. For example, the biological effect of the therapeutic amount may occur at and be measured at the cellular level by monitoring the hallmark characteristics of apoptosis, or the biological effect of the therapeutic amount may occur at and be measured at the system level, such as affecting levels of hormones or tumor disappearance. The biological effect of the therapeutic amount may occur at and be measured at the organism level, such as the alleviation of a symptom(s) or progression of a disease or condition in a subject. A therapeutically acceptable amount of a compound, composition or radiation dosage of the invention, regardless of the formulation or route of administration, may result in one or more biological responses in a subject. In the event that the compound or composition of the invention is subject to testing in an in vitro system, a therapeutically acceptable amount of the compound or composition may be viewed as that amount which gives a measurable response in the in vitro system of choice.

As used herein, the terms “treat”, “treating” and “treatment” include palliative, curative and prophylactic treatment.

As used herein, “measurable” means that an effect is both reproducible and significantly different from the baseline variability of the assay.
As used herein, “tumor shrinkage” refers to a measurable reduction in tumor size. The reduction may be measured by weighing an excised tumor mass, by weighing a particular volume of a tumor mass (i.e., a cube of 5 mm×5 mm×5 mm and calculating volume using a formula such as tumor volume (mm³)=π/6×length×width²), by microscopically or macroscopically measuring the height, width and depth of a tumor mass and/or by counting cells in a given sample. Typically, the measurements of a tumor are taken, treatment applied and additional measurements taken at later points or point in time and compared to the starting size of the tumor and/or compared to a control tumor receiving no treatment or other appropriate control tumors.

As used herein, “delayed tumor growth” refers to a measurable delay in tumor growth over a given period of time. The growth of the tumor may be measured by weighing an excised tumor mass, by weighing a particular volume of a tumor mass (i.e., a cube of 5 mm×5 mm×5 mm and calculating volume using a formula such as tumor volume (mm³)=π/6×length×width²), by microscopically or macroscopically measuring the height, width and depth of a tumor mass and/or by counting cells in a given sample. Typically, the measurements of a tumor are taken at a point in time, treatment applied and additional measurements taken over time and compared to the starting size of the tumor and/or compared to a control tumor receiving no treatment. A tumor showing delayed tumor growth is one that, as compared to a non-treated control tumor or other appropriate control tumor, is smaller in size and/or weight and/or contains a reduced number of viable cells.

As used herein, “tumor 4× growth delay” (TGD-4) refers to the difference in time (in days) between the time it takes a tumor subject to treatment to quadruple in volume as compared to the time it takes a control tumor to quadruple in volume.

As used herein, “cell proliferation” or “cellular proliferation” refers to those cells which undergo or attempt to undergo nuclear (mitotic or meiotic) and/or cellular (cytokinetic) division. Normal cell proliferation is used herein to describe those cells which successfully undergo and complete all stages of nuclear division as well as cellular division. Cancer cell proliferation is used herein to describe those cells which may successfully undergo and complete all stages of nuclear division as well as cellular division in an uncontrolled manner as compared to normal cells of the same tissue or culture, i.e., when the cellular division results in too many cells per unit area, undifferentiated cells, cells which show abnormalities in metabolism, appearance, ploidy or function, cells which attract and initiate unwanted angiogenesis, cells which secrete excess hormones or other metabolites and the like. Cancer cell proliferation is also used herein to describe those cells which undergo nuclear division(s) without cellular division.

“Decreased cancer cell proliferation” thus refers to a reduction in the number of tumor cells which successfully undergo and complete all stages of nuclear division as well as cellular division in an uncontrolled manner as compared to normal cells of the same tissue or culture, or a reduction in the number of tumor cells which undergo nuclear division(s) without cellular division. Decreased cancer cell proliferation also encompasses not only the reduction in the number of cells, but encompasses any reduction of the rate at which nuclear or cellular division proceeds as compared to normal cells.

As used herein, “decreased cancer cell survival” refers to the reduction of the number of viable cells in a given sample. Viability may be measured in a number of ways, including but not limited to, staining or application of dyes, measurement of O₂ uptake, CO₂ output, measurement of ATP and/or ADP, counting of cells in samples over time, measurement of DNA content and intactness, measurement of protein content and intactness, measurement of RNA content and intactness and the like.

As used herein, “cell cycle arrest” refers to the failure of a cell to progress through all stages of the cell cycle. The arrest may occur at any stage of the cell cycle: G₀, G₁, G₂, S or M.

Cell death and apoptosis are used herein in an interchangeable manner. As used herein, “cell death” or “apoptosis” refer to the programmed and deliberate death of a cell. Programmed cell death is the result of a series of controlled and orchestrated biochemical and physical reactions which terminate and dismantle a cell. The hallmarks of apoptosis include changes to the cellular membrane such as the loss of symmetry, integrity or attachment to other cells or surfaces, “blebbing” of the membrane in which vesicles are pinched off and released from the dying cell, fragmentation of chromosomal DNA, condensation of the chromatin, fragmentation of the nuclear membrane, changes in RNA patterns of expression and changes in protein expression. In many multicelluar organisms, macrophages often collect the cellular debris. In contrast, “necrosis” refers to the death of a cell that results from cellular injury or insult and does not proceed in a controlled manner.

As used herein, “enhanced” refers to the measurable increased and/or additive effect of one treatment upon a second treatment. The treatments may be applied simultaneously or separately. For example, the administration of a somatostatin or somatostatin analog may be used to increase the tumor shrinkage properties, delayed tumor growth properties, decreased cancer cellular proliferation properties, decreased cancer cellular survival properties, cell cycle arrest and/or increased cancer cell death properties of an external radiation treatment. In another example, the administration of a somatostatin or somatostatin analog may be used to increase the alleviation of excess hormone production properties and/or alleviation of any other biological complications properties resulting from the tumor, its growth and its effects upon surrounding and distant tissues as affected by an external radiation treatment.

As used herein, a “pansomatostatin” refers to a somatostatin agonist that selectively binds to at least three different somatostatin receptors and where the weakest binding affinity (Ki in nM) of the pansomatostatin to any SSTR receptor is no more than 100 times weaker than the strongest binding affinity for the same compound for at least 3 of the 5 different somatostatin receptor subtypes. Native somatostatin, which binds to all 5 receptor subtypes with relatively equal affinity is an example of a pansomatostatin. For example, a somatostatin agonist binding to SSTR1, SSTR2 and SSTR5 with affinities of 0.1 nM, 2.3 nM and 4.7 nM, respectively, and affinities for SSTR3 and SSTR4 of 112 nM and 572 nM, respectively, may be classified as a pansomatostatin.

What is meant by an SSTR1 receptor agonist (i.e., SSTR1 agonist) is a compound which has a high binding affinity (e.g., Ki of less than 100 nM or preferably less than 10 nM or less than 1 nM) for SSTR1 (e.g., as defined by the receptor binding assay in U.S. Pat. No. 7,084,117 incorporated herein by reference in its entirety). SSTR2, SSTR3, SSTR4 and SSTR5 receptor agonists are defined in a similar fashion as appropriate to each receptor and ligand.

What is meant by an SSTR1 receptor selective agonist is an SSTR1 receptor agonist that has a higher binding affinity at least 10× stronger (i.e., lower Ki) for SSTR1 than for another receptor, i.e., SSTR2, SSTR3, SSTR4 or SSTR5. SSTR2, SSTR3, SSTR4 and SSTR5 receptor selective agonists are defined in a similar fashion as appropriate to each receptor and ligand.

As used herein, “peripherial administration” includes all forms of administration of a compound or a composition comprising a compound of the instant invention. Examples of peripheral administration include, but are not limited to, oral, parenteral (e.g., intramuscular, intraperitoneal, intravenous or subcutaneous injection, implant and the like), nasal, vaginal, rectal, sublingual, inhalation or topical routes of administration, including transdermal patch applications, ointments, creams and the like.

As used herein, “radiosurgery” or “stereotactic surgery” refers to a non-invasive means of treating benign and/or malignant tissue or tumor growths by using directed beams of externally applied ionizing radiation. The ionizing radiation is administered in a dose suitable for irradiation of the target tissue, tumor or cancer. Radiosurgery is particularly useful in the ablation of tumors and other lesions which are not easily accessible by surgery such as, but not limited to, intra- and extra-cranial tumors such as pituitary adenomas. Cobalt-60 and X-rays are two common sources of radiation for use with this surgery. The radiation dose is usually measured in grays, where one gray (Gy) is the absorption of one joule per kilogram of mass.

The nomenclature used to define the peptides is that typically used in the art wherein the amino group at the N-terminus appears to the left and the carboxyl group at the C-terminus appears to the right. Where the amino acid has D and L isomeric forms, it is the L form of the amino acid that is represented unless otherwise explicitly indicated.

The somatostatin or somatostatin agonist compounds of the invention useful for enhancing the effects of externally applied radiation may possess one or more chiral centers and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilizing methods known to those skilled in the art. In addition, chiral intermediate compounds may be resolved and used to prepare chiral compounds of the invention.

The somatostatin or somatostatin agonist compounds of the invention useful for enhancing the effects of externally applied radiation may exist in one or more tautomeric forms. All tautomers and mixtures thereof are included in the scope of the present invention. For example, a claim to 2-hydroxypyridinyl would also cover its tautomeric form, α-pyridonyl. As used herein, 2-Nal refers to β-(2-naphthyl)alanine and D-2-Nal refers to the D form of this amino acid.

The pharmaceutically acceptable salts of the compounds of the invention which contain a basic center are, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. Compounds of the invention can also provide pharmaceutically acceptable metal salts, in particular non-toxic alkali and alkaline earth metal salts, with bases. Examples include the sodium, potassium, aluminum, calcium, magnesium, zinc and diethanolamine salts (Berge, S. M. et al., J. Pharm. Sci., 66:1-19 (1977); Gould, P. L., Int'l J. Pharmaceutics, 33:201-17 (1986); and Bighley, L. D. et al., Encyclo. Pharma. Tech., Marcel Dekker Inc, New York, 13:453-97 (1996).

The pharmaceutically acceptable solvates of the compounds of the invention include the hydrates thereof. Also included within the scope of the invention and various salts of the invention are polymorphs thereof. Hereinafter, compounds, their pharmaceutically acceptable salts, their solvates or polymorphs, defined in any aspect of the invention (except intermediate compounds in chemical processes) are referred to as “compounds of the invention”.

### EXAMPLES

A mouse GH3 xenograft model was used to assess the anti-proliferative effects of lanreotide with or without external radiation. Administration of lanreotide alone for 10 days resulted in moderate inhibition of tumor growth, thus validating the use of this model to assess the effects of somatostatin analogs on pituitary tumor cell proliferation. Lanreotide was well tolerated, as evidence by the continued growth and weight of the animals. The anti-proliferative effect of lanreotide was observed irrespective of whether the compound was administered daily or as a split-daily dose, suggesting that anti-proliferative effects depend on the absolute daily dose, not the dose regimen.

The results presented herein demonstrate that lanreotide co-administered with radiation was not radio-protective, i.e., the somatostatin analog did not reduce or negatively alter the response of GH3 tumors to radiation in vivo. Several tumor-bearing mice in the radiation and radiation plus lanreotide groups attained complete remission of tumors, a response which did not occur in the mice treated with lanreotide alone. Additionally, the data presented herein suggest that somatostatin analogs may play a role as radiation sensitizing and/or apoptosis enhancement agents useful in the treatment of pituitary and other tumors.

The skilled artisan would know and appreciate that other experiments may be designed and carried out to reach the determinations described in this invention. In these other experiments, the skilled artisan may use different types of mice or other model animals, different tumor cell lines, tissues or masses, different somatostatin agonist analogs, or even different sources of radiation to replicate the finding that somatostatin or somatostatin agonist analog compounds do not interfere with (i.e., are not radio-protective) the effects of externally applied radiation.

### In Vitro Experiments

### Cell Culture

Currently, there are no human pituitary GH-secreting cell lines that can be maintained in a differentiated state in vitro for sufficient time for colony formation assessment (Danila, D. C. et al., J. Clin. Endocrinol. Metab., 2001, 86:2976-8; Danila, D. C. et al., J. Clin. Endocrinol. Metab., 2000, 85:1180-7). As such, a rat GH3 pituitary tumor cell line was purchased from ATCC. The GH3 cells were maintained in DMED/F-12 medium (Gibco BRL, Grand Island, N.Y.) supplemented with 10% fetal calf serum, 100 units/ml penicillin and 100 μg/ml streptomycin in a 37° C. humidified incubator with a mixture of 95% air and 5% CO₂. All experiments were performed on exponentially growing cells with a doubling time of approximately 30 hours.

### In Vitro Clonogenic Assay

GH3 cells were detached from the cell culture support using a 0.05% trypsin-EDTA solution. The collected cells were counted, diluted in fresh growth medium and plated in triplicate in 60 mm Petri dishes (BD Biosciences, San Jose, Calif.) at dilutions of approximately 100-100,000 cells/dish. Lanreotide (Biomeasure, Inc., Milford, Mass., USA) was added to the diluted cells at final concentrations of 1-1000 nM. The cells were irradiated with 0-10 Gy at room temperature using a ¹³⁷Cs source with a dose rate of 300 cGy/min. Following exposure to lanreotide or gamma irradiation, the media was drained from the dish, the cells were washed twice with PBS (phosphate buffered saline) solution and the plates were filled with fresh growth medium. After incubation at 37° C. for 21 days, the cells were stained with 0.25% crystal violet. Colonies containing ≧50 cells were counted under a dissecting microscope and survival curves were generated. The plating efficiency (PE) was calculated as the percentage of cells plated that grew into colonies. The surviving fraction (SF) was defined as the fraction of cells surviving, i.e. number of colonies/(number of colonies plated×PE).

### Apoptosis and Cell Cycle Analysis

Apoptosis and cell cycle distribution was analyzed using a FACScan flow cytometer (BD Biosciences, San Jose, Calif.). The level of apoptosis was quantified by measuring the number of sub-diploid (sub-G1) cells. Briefly, GH3 cells were plated in 60 mm dishes at a density of approximately 500,000 cells/dish and treated with lanreotide and gamma irradiation. Approximately 48 to 168 hours after exposure, the cells were collected and washed with cold PBS supplemented with 5 mM EDTA. Cells were re-suspended in cold PBS-EDTA solution and fixed with cold 100% ethanol. After incubation for 30 minutes at room temperature, the cells were centrifuged and the pellet of cells was treated with 100 μg/ml of RNase A in a PBS-EDTA solution for 30 minutes at room temperature. Propidium iodide (PI) was added to a final concentration of 50 μg/ml and the DNA content was analyzed with a FACScan flow cytometer (BD Biosciences, San Jose, Calif.). The percentage of cells in the apoptotic sub-G1, G1, S, and G2/M phases was calculated.

### Dose Responses of GH3 Cells to Lanreotide and Gamma Irradiation

To identify doses of both lanreotide and irradiation that would produce a moderate level of cell killing, but not obscure a potential additive effect from combined therapy, the dose-response of GH3 cells to the treatment of both lanreotide and irradiation was characterized using a clonogenic assay.

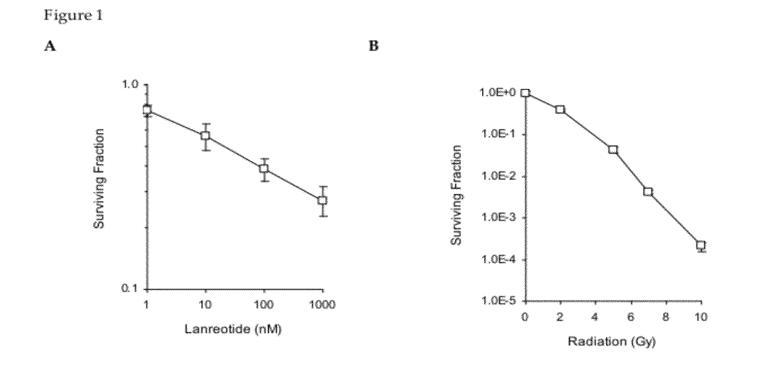

GH3 cells were plated in 60 mm tissue culture dishes and incubated overnight prior to treatment with the somatostatin agonist analog. Lanreotide was added to the cells to a final concentration of 1-1000 nM. After a 21 day incubation period in the presence of lanreotide the cells were stained and colonies with >50 cells were counted. As shown in FIG. 1A, treatment with lanreotide resulted in a dose-dependent decrease in GH3 cell colony forming units (CFU). Lanreotide at doses of 1, 10, 100, and 1000 nM resulted in cell survival rates of 75%, 56%, 39% and 27%, respectively. The IC50 (50% inhibition of cell growth) was 57 nM.

GH3 cells were plated in 60 mm tissue culture dishes and incubated overnight prior to exposure to radiation. The radiation survival curves are shown in FIG. 1B. The GH3 cells demonstrated a typical radiation dose-response survival curve with an initial shoulder at doses below 5 Gy and a straight line at higher doses. The surviving fraction at 2 Gy (a dose commonly used in daily fractionated radiotherapy and referred to as SF2) was 40%.

### Effect of Lanreotide on Radiation Response of GH3 Cells

GH3 cells were plated in tissue culture dishes and allowed to incubate overnight prior to treatment. At 48, 24 or 0 hours before radiation exposure, lanreotide was added to the cells at a final concentration of 100 nM or 1000 nM. At 48, 24 or 0 hours after the addition of the lanreotide, the GH3 cells were irradiated with 0-10 Gy at room temperature with a Cs-137 gamma irradiator. Following irradiation, cells with 24-hour or 0-hour pre-exposure to lanreotide were incubated in lanreotide-containing media for an additional 24 or 48 hours, respectively. After a total of 48-hour exposure, lanreotide-containing media was removed from the plates, the cells washed twice with PBS solution and fresh growth media added to the cells. Cells that were irradiated without exposure to lanreotide were also washed twice with PBS and supplied with fresh media. The cells were incubated for 21 days for colony formation.

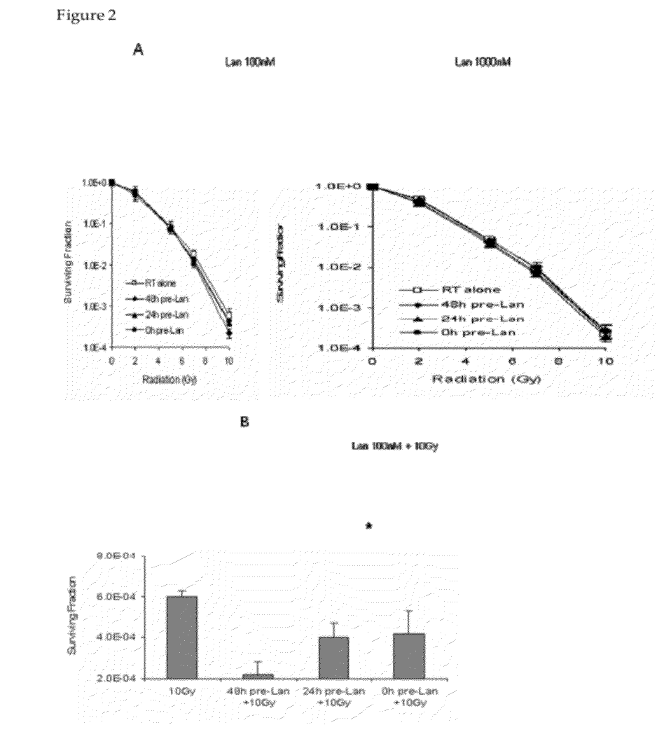

The radiation survival curves are shown in FIG. 2. Treatment with lanreotide alone at doses of either 100 nM or 1000 nM for 48 hours without radiation reduced clonogenic survival compared with untreated controls by 5-10%. Radiation alone without lanreotide produced a dose-dependent survival curve with a SF2 of 48-55%. Treatment with lanreotide at a dose of 100 nM for 48 hours either before (48 hours pre-lanreotide and 24 hours pre-lanreotide) or at the time of radiation (0 hour pre-lanreotide) produced survival curves that were slightly shifted downward and separated at doses of 7-10 Gy from the survival curve produced by radiation alone without lanreotide (FIG. 2A). These data indicate that the radiation response of GH3 cells was actually enhanced and not inhibited by lanreotide. The surviving fraction at 10 Gy was 0.0006, 0.00022, 0.00040 and 0.00042, respectively, for radiation alone, 48 hours pre-, 24 hours pre- and 0 hour pre-exposure to lanreotide (FIG. 2B). Treatment with 1000 nM lanreotide however, did not alter the shape and slopes of the radiation survival curves, indicating there was no radioprotective (or radiosensitization) effect under these experimental conditions (FIG. 2C).

### Effect of Lanreotide and Radiation on Apoptosis and Cell Cycle Distribution

GH3 cells were placed in 60 mm Petri dishes at a concentration of approximately 500,000 cells/dish and allowed to grow overnight. Lanreotide at 100 nM was added at 48 hours, 24 hours or immediately (0 hours) before irradiation. Cells were irradiated with 10 Gy gamma radiation at room temperature and collected 48, 72, 96 and 168 hours after exposure.

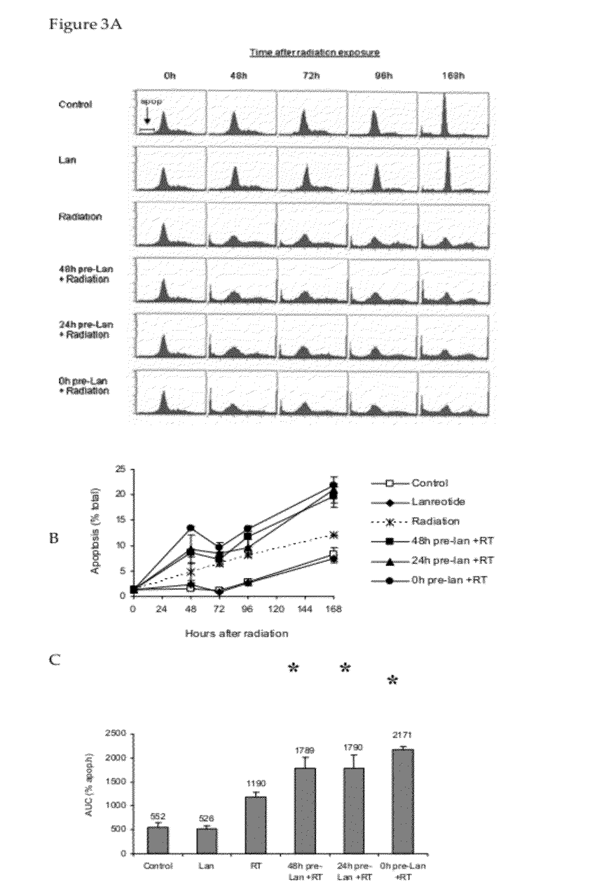

The DNA content of the cells was analyzed using a FACScan and the percentages of cells in the apoptotic sub-G1, G1, S and G2/M phases were calculated. As shown in FIG. 3A, untreated control cells showed a consistent cell cycle distribution over the course of 168 hours. The percentage of cells in sub-G1, G1, S and G2/M phases at 48 hours was 1.4±0.2%, 73.2±1.0%, 8.4±1.0% and 16.9±1.8%, respectively. Treatment with 100 nM lanreotide alone resulted in the sub-G1, G1, S and G2/M phase distribution of 2.28±0.3%, 73.8±1.1%, 7.72±0.8% and 16.2±0.5%, respectively. These data indicate that the cell cycle profile was not significantly affected by treatment with lanreotide except for a moderate increase in apoptotic sub-G1 cells from 1.4% to 2.28%.

Treatment with 10 Gy irradiation resulted in a decrease in the proportion of cells in G1 phase from 73.2% to 51.5% at 48 hours. Meanwhile, proportion of cells in the G2/M phase increased from 16.9% to 35.7% at 48 hours after irradiation; in addition, the cells were arrested at G2/M phase for up to 168 hours without release. The sub-diploid cell population, representing apoptotic cells, increased steadily following irradiation from a baseline of 1.4% to a peak of approximately 12% at 168 hours.

Combined treatment of GH3 cells with radiation and lanreotide produced a cell cycle profile that was similar to that seen in irradiated cells without lanreotide, except for an increase in apoptotic sub-G1 proportion. As shown in FIG. 3B, at 48 hours after radiation, the apoptotic sub-G1 cells increased from 4.9% for radiation alone to 8.6%, 9.3% and 13.4% for the combination of radiation with 48, 24 and 0 hours pre-exposure of lanreotide, respectively. These data represent an increase of apoptotic sub-G1 cells from 77%-173% as compared to radiation alone (P<0.01). At 168 hours after radiation, the sub-G1 cell fraction was 12% for radiation alone and 20-22% for radiation plus lanreotide, representing an increase of 67% to 83% (P<0.01). As can be seen in FIG. 3C, the accumulated distribution of apoptotic sub-G1 cells was significantly increased in cells that were treated with the combination of lanreotide and external radiation compared with externally applied radiation alone.

### Mouse Xenograft Tumor Model and Therapy

Male nude mice, 8 weeks old and 20-25 grams in body weight, were used in this study (Charles River Laboratories, Hollister, Calif.). Prior to experimentation the mice were tested and found to be negative for specific pathogens. The mice were maintained under specific-pathogen-free conditions and allowed to breed. Sterilized food and water were available ad libitum.

Tumors were initiated in the in the right flank of the mice by a subcutaneous injection of 5×10⁶ tumor cells suspended in 100 μl of a 1:1 mixture of Hank's solution and Matrigel (BD Biosciences, San Jose, Calif.). Each mouse received one inoculation injection. When the tumors reached an average size of 120 mm³ (80-200 mm³), the mice were randomly assigned to different treatment groups with 5-8 mice in each group. Lanreotide was administered to each mouse via subcutaneous injection. Dosages ranged from 1, 10, 100 or 1000 nM.

Local irradiation of individual tumors was carried out as follows. Unanesthetized tumor-bearing mice were placed in lead boxes, positioned so as to have the tumors protruding through a cut-out window at the rear of each box. The radiation dose was delivered using a Philips RT-250 200 kVp X-ray unit (12.5 mA; Half Value Layer, 1.0-mm Cu) at a dose rate of 138 cGy/min. The tumors were exposed to 150-250 cGy per fraction daily for 5 consecutive days as specified in each experiment.

Tumor volume was calculated using the formula: tumor volume (mm³)=π/6×length×width². The length and width of the tumors were measured with calipers before treatment and three times a week thereafter, until the tumor volume reached at least 4 times (4×) the pre-treatment volume. The tumor volume quadrupling (4×) time was determined by a best-fit regression analysis. The tumor growth delay (TGD) time (in days) is the difference between the tumor volume quadrupling time of treated tumors compared to the tumor volume quadrupling time of untreated control tumors. Both the tumor volume quadrupling time and tumor growth delay time were calculated for each individual animal and then averaged for each group. In some experiments, a complete response of tumors was recorded if a tumor shrunk to the point that it was not palpable at the end of the experiment. Body weight was measured twice a week.

### Statistical Analysis

The significance of differences between mean values obtained for the various study endpoints was calculated using an unpaired Student's t test.

### Results

### In Vivo Experiments

### Dose Responses of GH3 Tumors to Lanreotide

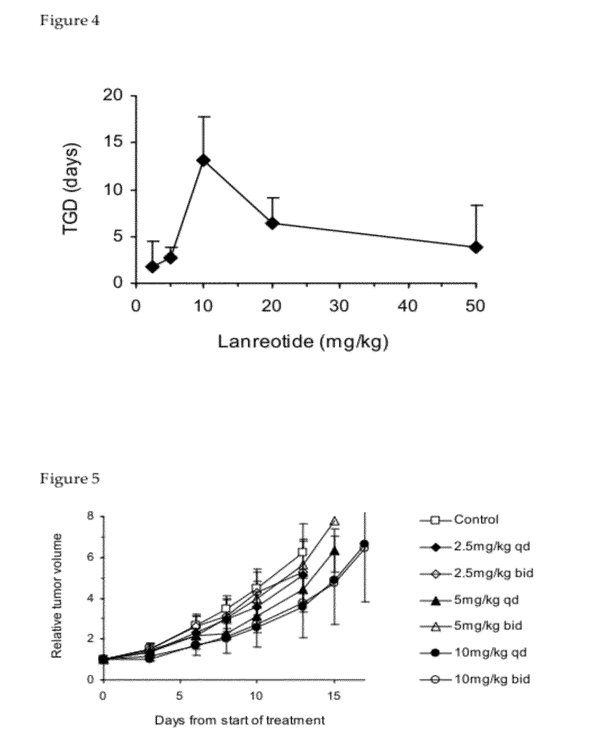

Groups of nude mice with established GH3 xenograft tumors were treated subcutaneously with 2.5, 5, 10, 20 or 50 mg/kg lanreotide daily for 5 days (Melen-Mucha, G. et al., Neoplasma, 2004, 51:319-24; Prevost, G. et al., Life Sci., 1994, 55:155-62). As shown in FIG. 4, the dose dependent effect of lanreotide on GH3 tumor growth followed a bell-shaped curve. The optimal dose resulting in the longest tumor growth 4× delay (13.1±4.7 days) occurred with a daily lanreotide dose of 10 mg/kg.

Lanreotide at all doses tested did not cause significant decrease in body weight compared to untreated control mice. Also, there was no notable change in the general appearance and daily activity of tumor-bearing mice treated with lanreotide.

Comparison of Lanreotide Dose Regimen of Once Daily Vs. Twice Daily

The effect of a single daily dose (qd) or two daily doses (bid) of lanreotide upon tumor growth was determined. GH3 tumor-bearing nude mice were injected subcutaneously with lanreotide at doses of 2.5, 5 or 10 mg/kg either once daily or twice daily (8 hour interval) for 5 days and tumor sizes measured. As shown in FIG. 5, lanreotide delivered once a day was as effective as lanreotide delivered twice daily in reducing tumor growth (p=0.3-0.9).

Lanreotide at 10 mg/kg once daily produced the longest tumor growth 4× delay (4.9±2.1 days) of all dose regimens studied (p<0.05). The delay resulting from the 10 mg/kg dose of lanreotide was significantly longer than the delay resulting from the administration of 5 mg/kg twice daily (1.1±3.1 days). In addition, the 5 mg/kg daily dose of lanreotide resulted in a longer tumor growth delay than did 2.5 mg/kg of lanreotide administered twice daily. These data suggest that one daily dose of lanreotide was at least as effective at delaying tumor growth as the same total dosage administered in two fractionated doses in one day. Further studies utilized the single daily dosing regimen.

### Combination Therapy of Lanreotide and Fractionated Radiation

To study the effect of lanreotide on tumor responses to radiation therapy, nude mice with established GH3 tumors were treated with one of the following:

A) 10 mg/kg lanreotide daily for 5 days;
B) local tumor irradiation daily for 5 consecutive days at doses of 250, 200 or 150 cGy/fraction/day;
C) a combination of lanreotide injected 20 minutes before local tumor radiation as above; or
D) sub-cutaneous injection of saline (0.005 ml/gram body weight) daily as an untreated control.

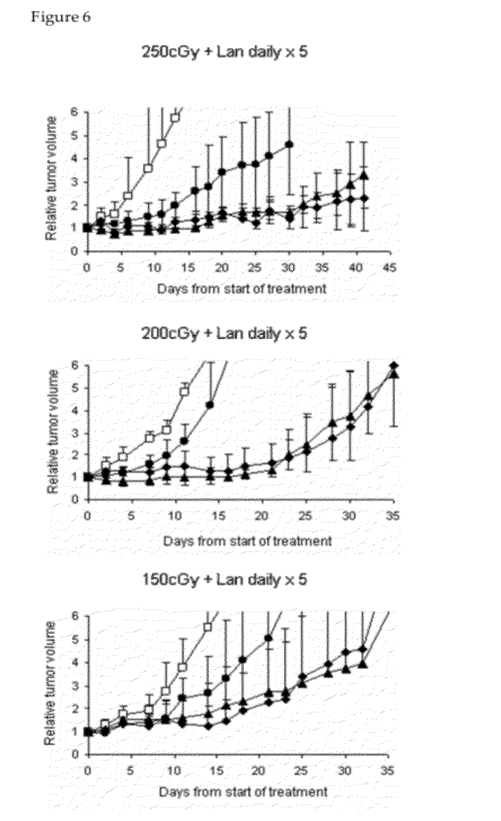

Data are shown in FIG. 6 (tumor growth curves). Lanreotide alone at a dose of 10 mg/kg moderately inhibited the growth of GH3 tumors with a 4× tumor growth delay time that ranged from 4.5 to 8.3 days (p=0.3˜0.06, compared to the relevant control groups). Fractionated local tumor irradiation alone significantly inhibited tumor growth and produced tumor growth delay times of 35.1±5.7 days for 250 cGy fractions, 21.7±5.5 days for 200 cGy fractions, and 16.7±1.7 days for 150 cGy fractions, respectively. The combination of lanreotide with radiation of 250, 200 or 150 cGy/fraction for 5 days inhibited tumor growth and produced the tumor growth delay times that were similar to radiation alone (p>0.05). Also, the combined treatment of lanreotide and fractionated radiation did not cause any further decrease in animal body weight compared to fractionated radiation therapy alone.

Pre-Administration of Lanreotide in Combination with Radiation Therapy

To determine the effect of pre-administration of lanreotide upon the effect of external irradiation on tumor growth, nude mice with GH3 xenograft tumors were treated with one of the following:

A) 10 mg/kg lanreotide daily for 10 days;
B) 150 cGy local tumor radiation daily for 5 consecutive days;
C) 10 mg/kg lanreotide for 5 days followed by combined administration of lanreotide and 150 cGy radiation daily for 5 days;
D) sub-cutaneous injection of saline (0.005 ml/gram body weight) daily for 10 days as an untreated control.

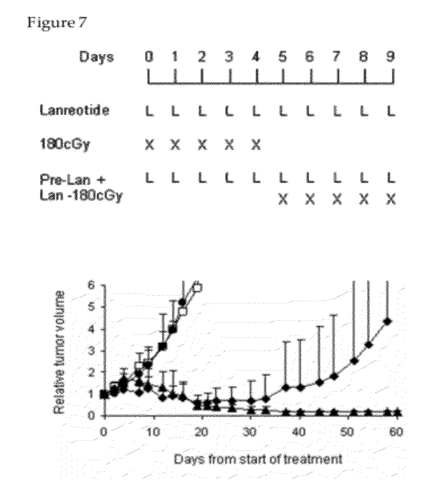

As shown in FIG. 7, lanreotide at a dose of 10 mg/k for 10 days moderately inhibited tumor growth (4×TGD, 8.3±8.3 days, p=0.06 vs. control). Local external tumor irradiation of 150 cGy inhibited tumor growth and gave a tumor growth delay time of 15.5.0±8.8 days (p<0.05 vs. control and lanreotide alone). The combination therapy of pre-administration of lanreotide and externally applied radiation resulted in a tumor growth delay time of 15.1±8.6 day, similar to that produced by radiation therapy alone (15.5±8.8 days) (p>0.05). Two mice from the irradiation and combination therapy groups exhibited complete tumor regression; upon termination of the study after 60 days, no tumor growth was observed. Lanreotide alone and in combination with external radiation did not result in any obvious signs of systemic toxicity as evidenced by the normal general appearance, skin reaction, body weight or activity levels of the mice.

### Discussion

Somatostatin analogs, such as lanreotide, vapreotide and octreotide, interact primarily with the somatostatin type-2 and type-5 receptors to reduce hormone production in neuroendocrine tumors, such as reducing the production of GH in pituitary somatotroph adenomas associated with acromegaly (see Ning, S., et al., Endocrine-Related Cancer, 2009, 16:1045-1055 and references cited therein). In addition to affecting hormone production, somatostatin analogs also exhibit anti-proliferative effects.

Observations by Landolt et al. (2000, supra) suggested that somatostatin analogs acted to protect cells from the effects of externally applied radiation. In contrast, the studies reported upon herein demonstrate that administration of lanreotide either before or during radiation had no effect upon cell survival, that is, was not radioprotective as suggested by Landolt. Studies described herein demonstrated that treatment with lanreotide, for example 100 nM for 48 hours prior to radiation, shifted survival curves downward and increased the apoptotic cell population at the higher doses of radiation. Such data suggests that somatostatin analogs such as lanreotide may act to synergistically enhance the radiation response of cancer cells.

Radiation and somatostatin analogs induce apoptosis in tumor cells via different signaling pathways; as such a combination of these two types of treatment may lead to a synergistic effect to increase or enhance apoptotic cell death. As shown herein, lanreotide enhanced radiation-induced apoptosis in GH3 pituitary tumor cells. Populations of cells exposed to 10 Gy radiation and 100 nM lanreotide exhibited an overall increase in sub-diploid cells (see Ning, S., et al., Endocrine-Related Cancer, 2009, 16:1045-1055 and references cited therein).

### Administration and Use

The somatostatin or somatostatin agonist compounds of this invention can be provided in the form of pharmaceutically acceptable salts. Examples of such salts include, but are not limited to, those formed with organic acids (e.g., acetic, lactic, maleic, citric, malic, ascorbic, succinic, benzoic, methanesulfonic, toluenesulfonic, or pamoic acid), inorganic acids (e.g., hydrochloric acid, sulfuric acid, or phosphoric acid), and polymeric acids (e.g., tannic acid, carboxymethyl cellulose, polylactic, polyglycolic, or copolymers of polylactic-glycolic acids). A typical method of making a salt of a peptide of the present invention is well known in the art and can be accomplished by standard methods of salt exchange. Accordingly, the TFA salt of a peptide of the present invention (the TFA salt results from the purification of the peptide by using preparative HPLC, eluting with TFA containing buffer solutions) can be converted into another salt, such as an acetate salt, by dissolving the peptide in a small amount of 0.25 N acetic acid aqueous solution. The resulting solution is applied to a semi-prep HPLC column (Zorbax®, 300 SB, C-8). The column is eluted with: (1) 0.1N ammonium acetate aqueous solution for 0.5 hours; (2) 0.25N acetic acid aqueous solution for 0.5 hours; and (3) a linear gradient (20% to 100% of solution B over 30 minutes) at a flow rate of 4 ml/min (solution A is 0.25N acetic acid aqueous solution; solution B is 0.25N acetic acid in acetonitrile/water, 80:20). The fractions containing the peptide are collected and lyophilized to dryness.

As is well known to those skilled in the art, the known and potential uses of somatostatin agonist compounds activity is varied and multitudinous, thus the administration of the compounds of this invention for purposes of eliciting an agonist effect can have the same effects and uses as somatostatin itself.

The dosage of active ingredient in the somatostatin or somatostatin agonist compounds compositions of this invention may be varied as necessary to obtain the optimum dosage for administration in conjunction with externally applied radiation; however, it is necessary that the amount of the active ingredient be such that a suitable dosage form is obtained. The selected dosage depends upon the desired therapeutic effect, on the route of administration and on the duration of the treatment and may also depend upon the target dose of externally applied radiation. In general, an effective dosage for the activities of this invention is in the range of 1×10⁻⁷ to 200 mg/kg/day, preferably 1×10⁻⁴ to 100 mg/kg/day which can be administered as a single dose or divided into multiple doses as needed to enhance the effects of externally applied radiation.

The somatostatin or somatostatin agonist compounds of this invention can be administered by oral, parenteral (e.g., intramuscular, intraperitoneal, intravenous or subcutaneous injection, or implant), nasal, vaginal, rectal, sublingual or topical routes of administration and can be formulated with pharmaceutically acceptable carriers to provide dosage forms appropriate for each route of administration. The external radiation can be provided in any acceptable form to a patient as a whole body treatment or a localized treatment. The external radiation may be electromagnetic or particulate including cobalt therapy and can include other forms of ionizing radiation such as X-rays, γ-rays, β-rays, ultraviolet light, near ultra-violet light and other sources of radiation including, for example, α-mesons.

Solid dosage forms for oral administration of somatostatin or somatostatin agonist compounds useful in the practice of this invention include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than such inert diluents, e.g., lubricating agents such as magnesium stearate. In the case of capsules, tablets and pills, the dosage forms may also comprise buffering agents. Tablets and pills can additionally be prepared with enteric coatings.

Liquid dosage forms for oral administration include pharmaceutically acceptable emulsions, solutions, suspensions, syrups, the elixirs containing inert diluents commonly used in the art, such as water. Besides such inert diluents, compositions can also include adjuvants, such as wetting agents, emulsifying and suspending agents, and sweetening, flavoring and perfuming agents.

Preparations according to this invention for parenteral administration of somatostatin or somatostatin agonist compounds include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. Preparations may be sterilized by, for example, filtration through a bacteria-retaining filter, by incorporating sterilizing agents into the compositions, by irradiating the compositions, or by heating the compositions. Preparations can also be manufactured in the form of sterile solid compositions which can be dissolved in sterile water or some other sterile injectable medium immediately before use.

Compositions for rectal or vaginal administration are preferably suppositories which may contain, in addition to the active substance, excipients such as cocoa butter or a suppository wax. Compositions for nasal or sublingual administration are also prepared with standard excipients well known in the art. Further, a somatostatin or somatostatin agonist compound useful in the practice of this invention can be administered in a sustained release composition such as those described in the following patents and patent applications. U.S. Pat. No. 5,672,659 incorporated herein by reference in its entirety for teachings directed to sustained release compositions comprising a bioactive agent and a polyester. U.S. Pat. No. 5,595,760 incorporated herein by reference in its entirety for teachings directed to sustained release compositions comprising a bioactive agent in a gelable form. U.S. Pat. No. 5,821,221 incorporated herein by reference in its entirety for teachings directed to polymeric sustained release compositions comprising a bioactive agent and chitosan. U.S. Pat. No. 5,916,883 teaches sustained release compositions comprising a bioactive agent and cyclodextrin.

What is claimed is:

1. A method of treating a pituitary adenoma, breast cancer, prostate cancer or a neuroendocrine tumor in a subject in need thereof, said method comprising the administration of a therapeutically effective amount of somatostatin, or a somatostatin agonist analog or pharmaceutically acceptable salt thereof, in combination with a therapeutically effective amount of externally administered radiation therapy to treat said pituitary adenoma, breast cancer, prostate cancer or neuroendocrine tumor in said subject in need thereof.

2. The method according to claim 1, wherein said somatostatin agonist analog or pharmaceutically acceptable salt thereof, is an SSTR2 somatostatin agonist.

3. The method according to claim 2, wherein said somatostatin agonist analog or pharmaceutically acceptable salt thereof, is an SSTR2 selective somatostatin agonist.

4. The method according to claim 3, wherein said somatostatin agonist analog, or pharmaceutically acceptable salt thereof, is selected from the group consisting of lanreotide (SEQ ID NO:1), octreotide (SEQ ID NO:2) or vapreotide (SEQ ID NO:3).

5. The method according to claim 1, wherein said somatostatin agonist analog or pharmaceutically acceptable salt thereof, is a pansomatostatin agonist.

6. The method according to claim 1, wherein said externally administered radiation is stereotactic radiosurgery.

7. The method according to claim 1, wherein said treatment results in tumor shrinkage, delayed tumor growth, decreased tumor growth, decreased cancer cell proliferation, decreased cancer cell survival, increased cancer cell cycle arrest, increased cancer cell apoptosis or alleviation of symptoms associated with said pituitary adenoma.

8. The method according to claim 7, wherein cancer cell apoptosis is increased.

9. The method according to claim 1, wherein said somatostatin, somatostatin agonist analog, or pharmaceutically acceptable salt thereof, is administered prior to said external radiation.

10. The method according to claim 9, wherein said somatostatin, somatostatin agonist analog, or pharmaceutically acceptable salt thereof, is administered 1 to 7 days prior to said external radiation therapy.

11. The method according to claim 9, wherein said somatostatin, somatostatin agonist analog, or pharmaceutically acceptable salt thereof, is administered 48 hours prior to said external radiation therapy.

12. The method according to claim 9, wherein said somatostatin, somatostatin agonist analog, or pharmaceutically acceptable salt thereof, is administered 24 hours prior to said external radiation therapy.

13. The method according to claim 9, wherein said somatostatin, somatostatin agonist analog, or pharmaceutically acceptable salt thereof, is administered immediately prior to said external radiation therapy.

14. The method according to claim 1, wherein said somatostatin, somatostatin agonist analog, or pharmaceutically acceptable salt thereof, is administered concomitantly with said external radiation.

15. The method according to claim 1, wherein said somatostatin, somatostatin agonist analog, or pharmaceutically acceptable salt thereof, is administered after said external radiation.

16. The method according to claim 15, wherein said somatostatin, somatostatin agonist analog, or pharmaceutically acceptable salt thereof, is administered 1 to 7 days after said external radiation therapy.

17. The method according to claim 15, wherein said somatostatin, somatostatin agonist analog, or pharmaceutically acceptable salt thereof, is administered 48 hours after said external radiation therapy.

18. The method according to claim 15, wherein said somatostatin, somatostatin agonist analog, or pharmaceutically acceptable salt thereof, is administered 24 hours after said external radiation therapy.

19. The method according to claim 15, wherein said somatostatin, somatostatin agonist analog, or pharmaceutically acceptable salt thereof, is administered immediately after said external radiation therapy.

20. (canceled)

21. The method according to claim 1 wherein said pituitary adenoma is selected from the group consisting of ACTH-secreting adenomas, prolactin secreting adenomas, GH secreting adenomas and non-GH-secreting adenomas.

22. The method according to claim 21, wherein said subject suffers from acromegaly.


SEQUENCE LISTING (3 sequences)

SEQ ID NO: 1 - protein, 8 aa, Artificial Sequence
  Synthetic somatostatin agonist
XCYXKVCT

SEQ ID NO: 2 - protein, 8 aa, Artificial Sequence
  Synthetic somatostatin agonist
XCFXKTCT

SEQ ID NO: 3 - protein, 8 aa, Artificial Sequence
  Synthetic somatostatin analog
XCYCLVCW